(19) United States
(12) Patent Application Publication
(10) Pub. No.: US 2018/0185530 A1
(43) Pub. Date: Jul. 5, 2018

(54) SURFACE TREATMENT DEVICE

(71) Applicant: KONINKLIJKE PHILIPS N.V., EINDHOVEN (NL)

(72) Inventors: CORNELIS REINDER RONDA, EINDHOVEN (NL); JEAN-PAUL JACOBS, EINDHOVEN (NL); JAN FREDERIK SUIJVER, EINDHOVEN (NL); WEIZHONG CHEN, EINDHOVEN (NL); JEAN BART BLEEKER, EINDHOVEN (NL); TIMOTHY VAN DER GRAAF, EINDHOVEN (NL); JOHAN MARRA, EINDHOVEN (NL); ACHIM GERHARD ROLF KOERBER, EINDHOVEN (NL); RAINER HILBIG, EINDHOVEN (NL)

(21) Appl. No.: 15/736,992

(22) PCT Filed: Jun. 30, 2016
(86) PCT No.: PCT/EP2016/065450

§ 371 Date: Dec. 15, 2017

(30) Foreign Application Priority Data
Jun. 30, 2015 (CN) PCT/CN2015/082929
Aug. 11, 2015 (EP) 15180494.5

(51) Int. Cl. A61L 2/14; A61L 2/26; A47L 7/00

(52) U.S. Cl. CPC A61L 2/14; A61L 2202/11; A47L 7/0085; A61L 2/26

(57) ABSTRACT

A surface treatment device (100) is disclosed that comprises a conduit (110) including an air inlet (105) and an air outlet (115). The conduit comprises a reactive particles generator (130) for generating reactive particles from air and arranged to subject the surface to the generated reactive particles. The reactive particles generator (130) is also used for generating an air flow from the air inlet to the air outlet through the conduit.

### FIELD OF THE INVENTION

The present invention relates to treating a surface contaminated with micro-particles such as pollen and/or micro-organisms such as mites.

### BACKGROUND OF THE INVENTION

Air purification devices are commonly employed to filter air in closed environments, e.g. in domestic or commercial dwellings. Such air purification serves a number of purposes, e.g. odor control and allergen filtration from the treated air. Air purifiers are particularly effective in capturing gaseous compounds and particles such as PM 2.5 particles but are less effective in capturing larger micro-particles, such as pollen particles, as the weight of such particles typically causes such particles to precipitate on surfaces, where they can cause severe allergic reactions, e.g. asthmatic attacks in children playing in contact with such surfaces. Other harmful allergens include micro-organisms, e.g. dust-dwelling micro-organisms such as mites, which may be particularly problematic in bedding, e.g. pillows, duvets, blankets and mattresses.

Such larger micro-particles or micro-organisms may be effectively neutralized, e.g. decomposed using reactive particles, such as created in non-thermal plasmas.

WO 2012/104089 A1 discloses a floor cleaning machine, in particular a vacuum cleaner or a scrubber for cleaning a floor. The floor cleaning machine comprises an integrated plasma applicator for applying a non-thermal atmospheric plasma. However, such plasma generation causes the generation of harmful compounds as side products, such as ozone (O₃) and NO₂.

DE202008008729U1 discloses a device for cleaning objects such as mattresses. A blower is used to take in ambient air, mix it with plasma and subject a surface to the mixture.

US2005000054A1 discloses a vacuum cleaner having an ion generator. An electrically driven fan is used to draw in dust. Inside the vacuum cleaner an ion generator is present which kill floating germs in the air stream.

U.S. Pat. No. 5,236,512A discloses a method and apparatus for cleaning surfaces with plasma. Suction and supply devices are used to provide a gas mixture to a reaction chamber in order to eliminate contaminants from a surface.

U.S. Pat. No. 8,267,884B1 discloses a wound treatment apparatus. The apparatus contains a plasma generating device for producing a flow of gas to treat wounds with. A compressor is used to increase pressure to generate an air flow from the device towards a patient.

### SUMMARY OF THE INVENTION

The present invention seeks to provide a surface treatment that can effectively neutralize surface allergens without producing harmful amounts of side products. The invention is defined by the independent claims. The dependent claims define advantageous embodiments.

According to an embodiment of the invention, there is provided a surface treatment device comprising a conduit including an air inlet for contacting a surface, the conduit comprising a reactive particles generator for generating reactive particles from air and arranged to subject, e.g. directly subject, the surface to the generated reactive particles, an air outlet, and an air flow generator for generating an air flow from the air inlet to the air outlet through the conduit.

According to an embodiment of the invention, the air flow generator is configured to generate a net air flow velocity of 1 m/s or less. It has been surprisingly found that by guiding an air flow from the surface into the surface treatment device at such low velocities, an effective neutralization of surface allergens, i.e. micro-particles such as pollen or micro-organisms such as (dust) mites can be achieved without producing levels of side products such as ozone and NO₂ that are harmful. This compares favorably to e.g. plasma generating air purifiers than typically operate air flows in excess of 100 m³/hour, which corresponds to much higher air flow velocities, making air purifiers substantially noisier than the surface treatment device of the present invention. Effective neutralization of such surface allergens can be achieved by treating a surface area under treatment for a short period of time only, i.e. no more than several seconds, thus making the surface treatment device easy to use. Moreover, the low air velocity ensures low power consumption of the surface treatment device, which is desirable in terms of reducing the carbon foot print as well compliance with regulations designed to enforce such reduction.

According to an embodiment of the invention, the air flow generator is adapted such that the air flow is in the range of 0-10 m³/hour. For example, the air inlet may have an inlet area such that a net surface air flow through the air inlet is in the range of 0-10 m³/hour. This is substantially lower than the air flow through an air purifier for instance, which therefore translates to a substantial reduction in generation of harmful side products.

In a particularly advantageous embodiment, the net surface air flow is zero. In this embodiment, air turbulence may be created at the surface to be treated, with the reactive particles injected into the turbulent air to neutralize surface allergens at the surface. The higher velocity of the reactive particles compared to the net air flow velocity ensures that the reactive particles can travel opposite to the direction of the net air flow and can penetrate the surface to be treated, e.g. a carpet, soft furnishing, mattress and so on, to effectively neutralize surface allergens without requiring a net surface air flow. In an embodiment, the air inlet and the air outlet are located such that a net air flow at the surface is zero.

The reactive particles generator may for instance be an ionization device or a plasma generator. Particularly preferred is a dielectric barrier discharge plasma generator or a corona discharge generator. A corona discharge generator may further act as (part of) the air flow generator by generating ionic wind resulting from the corona discharge. In such an embodiment the corona discharge device has a double functionality. Its first function is to generate the air flow from the air inlet to the air outlet. Its second function is to generate reactive particles, e.g. plasma, from air. Thus, by using a corona discharge device, only a single component is needed for generating air flow and reactive particles instead of two separate components as disclosed by the prior documents. This obviates the need for additional components in the surface treatment device, thus reducing its cost.

According to an embodiment of the invention, the reactive particles generator comprises a corona wire. The conduit and the corona wire are adapted such that particles on the surface are directly exposed to the generated reactive particles at the corona wire when the surface is subjected to the surface treatment device. For example, the conduit is shaped and the corona wire is located in the conduit such that particles on the surface are directly exposed to the generated reactive particles at the corona wire when the surface is subjected to the surface treatment device.

According to an embodiment of the invention, the reactive particles generator further comprises a collector electrode. The conduit and the collector electrode are adapted to generate a vortex inside the conduit when the air flow is generated such that particles on the surface are transported towards generated plasma at the counter electrode via the vortex when the surface is subjected to the surface treatment device. For example, the conduit is shaped and the collector electrode is located in the conduit such that a vortex is generated inside the conduit when the air flow is generated such that particles on the surface are transported towards generated plasma at the counter electrode via the vortex when the surface is subjected to the surface treatment device. For example, the conduit features an isolating divider supporting the collector electrode, the isolating divider being positioned inside the conduit and adapted for generating the vortex.

Preferably, the surface treatment device further comprises an ozone neutralizing element in the air flow, such as in the conduit between the reactive particles generator and the air outlet or in the air outlet. The ozone element may be located downstream from the reactive particles generator. An example of such an ozone neutralizing element is an active carbon containing element. As active carbon reacts with ozone, this further reduces the amount of ozone produced by the surface treatment device. Alternatively, catalysts that decompose ozone may be used in the ozone neutralizing element.

The surface treatment device may be adapted to generate an air flow between a discrete air inlet and air outlet. Alternatively, the air inlet forms at least part of the air outlet or the air outlet forms at least part of the air inlet, e.g. in an air circulation or air turbulence-based surface treatment device comprising a single opening.

The air flow generator may comprise or may further comprise a fan for forcing air from the air inlet to the air outlet.

In an embodiment, the surface treatment device comprises a removable head including the air inlet and the reactive particle generator. This allows for the surface treatment device to be operated without the reactive particle generator, e.g. by using a different head as well as for replacement of the reactive particle generator by providing a replacement head, thus obviating the need to replace the entire surface treatment device in case of a reactive particle generator reaching its end of life.

The surface treatment device may be but is not limited to a vacuum cleaning device or a mattress cleaning device.

The surface treatment device may be a robotic surface treatment device, e.g. a robotic cleaning device.

Further, a method for neutralizing allergens on a surface is presented, comprising: generating an air flow from a surface; generating reactive particles from air; subjecting the surface to the reactive particles thereby neutralizing allergens present on the surface. Generating the air flow is performed by generating an ionic wind and subjecting the surface to the ionic wind. According to an embodiment, the generated ionic wind has an air flow velocity of 1 m/s or less.

These and other aspects of the invention will be apparent from and elucidated with reference to the embodiments described hereinafter.

### BRIEF DESCRIPTION OF THE DRAWINGS

Embodiments of the invention are described in more detail and by way of non-limiting examples with reference to the accompanying drawings, wherein:

FIG. 1 schematically depicts an embodiment of a surface treatment device;

FIG. 2 schematically depicts another embodiment of a surface treatment device;

FIG. 3 schematically depicts yet another embodiment of a surface treatment device;

FIG. 4 schematically depicts yet another embodiment of a surface treatment device; and

FIG. 5 schematically depicts yet another embodiment of a surface treatment device.

FIG. 6 schematically depicts an embodiment of an asymmetric ionic wind device used to generate air flow and generate reactive particles

FIG. 7 depicts the geometry of an embodiment of an asymmetric ionic wind device (measures in mm) according to an embodiment of the invention

FIG. 8 illustrates the distribution of electric potential in an asymmetric ionic wind device according to an embodiment of the invention

FIG. 9 illustrates the positive ion density u in an asymmetric ionic wind device according to an embodiment of the invention

FIG. 10 illustrates the ion current density in an asymmetric ionic wind device according to an embodiment of the invention

FIG. 11 illustrates that the electric field strength magnitude at the collector of an asymmetric ionic wind device according to an embodiment of the invention

FIG. 12 is a vector plot of the “ionic wind” volume force in an asymmetric ionic wind device according to an embodiment of the invention which is driving the air flow

FIG. 13 is a vector plot of the air velocity in an asymmetric ionic wind device according to an embodiment of the invention

FIG. 14 is a vector plot of the air velocity in a symmetric ionic wind device according to an embodiment of the invention

### DETAILED DESCRIPTION OF EMBODIMENTS

It should be understood that the Figures are merely schematic and are not drawn to scale. It should also be understood that the same reference numerals are used throughout the Figures to indicate the same or similar parts.

Throughout the description reference is made to “reactive particles”. This may refer to plasma or another substance that can disinfect particles such as allergens.

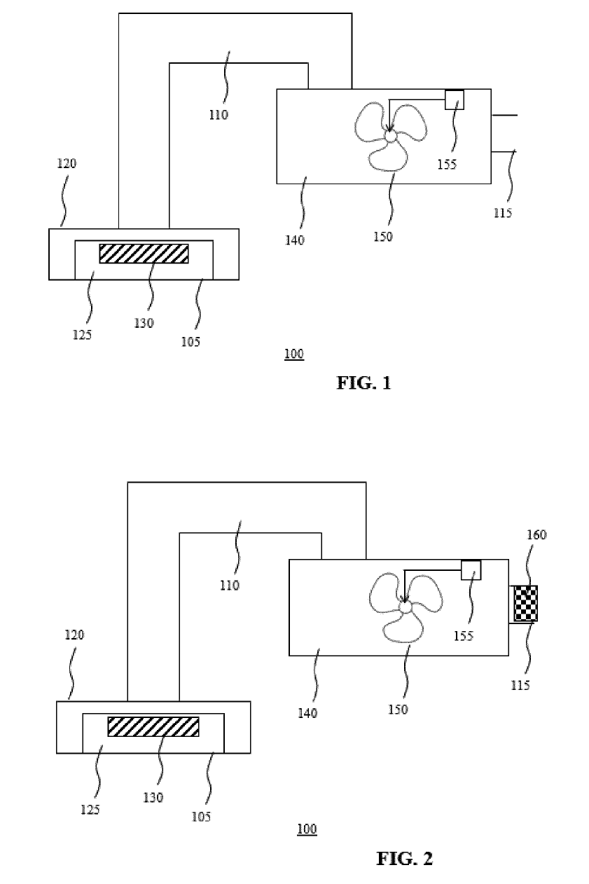

FIG. 1 schematically depicts a surface treatment device 100 according to an embodiment. The surface treatment device 100 comprises a conduit 110 located in between an air inlet 105 and an air outlet 115 and includes an air flow generator 150 for generating an air flow from the inlet 105 to the outlet 115. The conduit typically includes an enclosure or treatment chamber 125 in which a reactive particles generator 130 such as an ionization device or a plasma generating device is located. The plasma generating device typically is a non-thermal plasma generating device and employs a dielectric barrier discharge (DBD) operation, for example, as DBD generated plasmas are capable of decomposing allergens such as pollen or other relatively large micro-particles (i.e. larger than PM 2.5 particles) and micro-organisms ranging from viruses, bacteria and (dust) mites at second timescales. Alternatively, the plasma generating device may employ corona discharge operation, which may also be used to generate the air flow through the conduit 110 based on the well-known principle of ionic wind. In such an embodiment, the reactive particles generator 130 also acts as (part of) an air flow generator 150, which will be described in more detail below. The plasma generating device may be configured to generate plasma inside the device or around an outer surface of the device, e.g. in a volume of air around the outer surface(s) of the plasma generating device, such as in a volume of air having a thickness, i.e. extending from the outer surface(s) of the plasma generating device, of several mm, e.g. 1-5 mm or 1-2 mm.

The conduit 110 may include or may be in fluid connection with a compartment 140 housing an air flow generator 150, here depicted as a fan. It should be understood that a fan is shown by way of non-limiting example only and that any suitable air flow generator 150 may be employed. A particularly suitable fan-less alternative is an air flow generator 150 based on ionic wind, in which case the air flow generator 150 may be embodied at least in part by the reactive particles generator 130. This electrohydrodynamic effect is commonly referred to as ion wind or ionic wind, and provides a fan-less embodiment of the surface treatment device 100 that is particularly quiet in operation. As such air flow generators are well known per se, they will not be explained in further detail for the sake of brevity only. For examples, reference is made to the article “Ionic Winds: A New Frontier for Air Cooling” at http://www.electronics-cooling.com/2012/03/ionic-winds-a-new-frontier-for-air-cooling/.

The air flow generator 150 may be controlled by a controller 155 to control the air flow rate produced by the air flow generator 150. The surface treatment device 100 may be adapted to operate at a fixed air flow rate or alternatively may be adapted to operate at an adjustable air flow rate. The air flow rate may be adjustable by a user, e.g. by the inclusion of a user interface (not shown) in the surface treatment device 100 that allows its user to adjust the air flow rate. Alternatively, the surface treatment device 100 may include a sensor (not shown) for sensing the air quality of the air influx through the air inlet 105. This sensor may be located in any suitable location, e.g. upstream from the reactive particles generator 130, that is, in between the reactive particles generator 130 and the air inlet 105, such as in the treatment chamber 125 or outside the treatment chamber 125. The controller 155 may be responsive to a sensor signal generated by the air quality sensor such that the air flow rate is adjusted in accordance with the sensed air quality.

The air flow generator 150 is adapted to generate a net air flow velocity through the air inlet 105 of 1 m/s or less. Such low speed air flows allow the surface treatment device 100 to be operated quietly whilst still achieving effective neutralization of the surface allergens by the reactive particles generated by the reactive particles generator 130. Preferably, the net air flow through the air inlet 105 is in the range of 0-10 m³/hour. This for instance may be achieved by dimensioning the inlet area of the air inlet 105 and the air flow generator 150 accordingly and/or by configuring the controller 155 to operate the air flow generator 150 within this range of air flow rates. For example, for a surface treatment device 100 operating a net air flow rate of 10 m³/hour with an air flow velocity of no more than 1 m/s, the air inlet 105 would typically have an inlet area of at least 28 cm².

By operating the air flow generator 150 to generate a net air flow in the range of 0-10 m³/hour, the reactive particles generator 130 may be operated in a low energy mode, such that a relatively small amount of reactive particles, e.g. plasma, is generated per unit time, thus limiting the production of harmful reaction products such as ozone and NO₂. The relatively modest air flow rate further increases the dwell time of allergens in the treatment chamber 125, such that the allergens typically reside in the treatment chamber 125 for several seconds, which increases the effectiveness of the allergen decomposition by the reactive particles, e.g. ions or plasma radicals.

The treatment chamber 125 may be located in any suitable location in fluid connection with or as part of the conduit 110. In FIG. 1, the treatment chamber 125 is shown by way of non-limiting example as part of a removable head unit 120 of the surface treatment device 100, which head unit 120 may be secured on the conduit 110 in any suitable manner. For example, the conduit 110 may comprise a flexible or rigid tube having an end portion with a securing member and the removable head unit 120 may comprise a tube portion with a further securing member adapted to engage with the securing member to secure the removable head unit 120 onto the end portion. Such securing arrangements are well-known per se and it is noted that any suitable securing arrangement, i.e. any suitable securing member and further securing member may be used.

The surface treatment device 100 in some embodiments may contain a collection device, e.g. a dust bag, dust container or the like, in between the conduit 110 and the air outlet 115 to collect dust and other particles collected through the air inlet 105. However, in some other embodiments, such a collection device is omitted, in particular in embodiments in which the air flow rate (suction) generated by the air flow generator 150 is insufficient to suck dust into the conduit 110, such that only small micro-organisms and/or micro-particles are inactivated by the air flow generator 150.

FIG. 2 schematically depicts a surface treatment device 100 according to another embodiment. The surface treatment device 100 in FIG. 2 is largely identical to the surface treatment device 100 in FIG. 1 such that features of the surface treatment device 100 already described in the detailed description of FIG. 1 will not be described again for the sake of brevity. The surface treatment device 100 in FIG. 2 differs from the surface treatment device 100 in FIG. 1 in that the surface treatment device 100 further comprises an ozone neutralizing element 160 such as an active carbon containing element, e.g. an active carbon filter in the air flow downstream from the reactive particles generator 130 in order to filter out unwanted side products generated in the generation of the reactive particles by the reactive particles generator 130, most notably ozone. Other suitable embodiments of such an ozone neutralizing element 160 will be immediately apparent to the skilled person, such as a catalyst-based ozone neutralizing element 160 for decomposing ozone.

The ozone neutralizing element 160 may be located in any suitable location downstream from the reactive particles generator 130, e.g. in the conduit 110 or in the air outlet 115. The ozone neutralizing element 160 is preferably located in a location that is easily accessible by the user of the surface treatment device 100 to facilitate replacement of the ozone neutralizing element 160, e.g. an active carbon-containing element when necessary, such as in or over the air outlet 115, in or over an opening in the compartment 140 to which the conduit 110 removably connects, and so on.

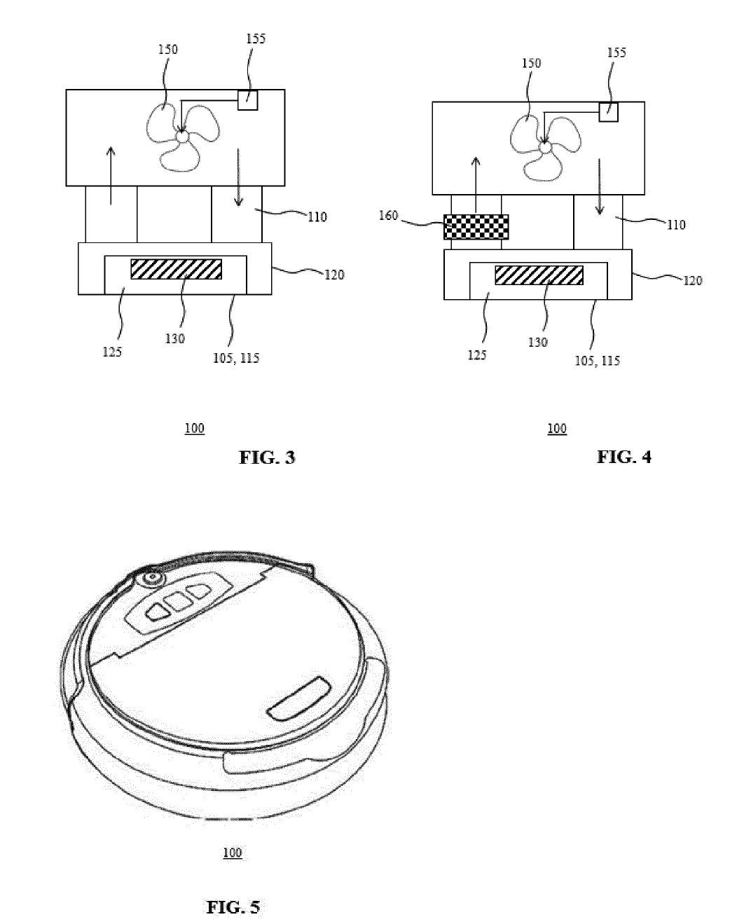

FIG. 3 schematically depicts another embodiment of the surface treatment device 100 in which the air inlet 105 and the air outlet 115 are comprised by a single opening, or at least in part share the same opening. In this embodiment, the conduit 110 creates a recirculation of air around the reactive particles generator 130 in the treatment chamber 125, e.g. air turbulence around the reactive particles generator 130, which may further increase the dwell time of allergens in the treatment chamber 125 as the allergens pass the treatment chamber 125 twice, i.e. when entering the surface treatment device 100 through the air inlet 105 and prior to exiting the surface treatment device 100 through the air outlet 115. Alternatively, the air inlet 105 and air outlet 115 may be located adjacent to each other, both in fluid connection with the treatment chamber 125.

In this embodiment, the net surface air flow can be zero. The operation of the surface treatment device 100 with low air flow velocity and a zero net surface air flow not only ensures quiet operation of the surface treatment device 100 but furthermore facilitates deep penetration of the surface by the reactive particles generated by the reactive particles generator 130 due to the fact that the escape velocity of the reactive particles from the reactive particles generator 130 typically is (much) larger, e.g. orders of magnitude larger, than the air flow velocity generated by the air flow generator 150, such that the reactive particles can travel against the direction of air flow generated by the air flow generator 150. This therefore not only ensures effective neutralization of allergens at the surface to be treated but also facilitates neutralization of allergens, e.g. dust mites, below the surface as the reactive particles can penetrate the surface and travel into the object comprising the surface, for example a bedding object such as a pillow or mattress, a soft furnishings object such as a sofa, couch, chair or the like, a rug or carpet, and so on.

FIG. 4 schematically depicts the surface treatment device 100 according to FIG. 3 further comprising an ozone neutralizing element 160 such as an active carbon-containing element or catalyst-based element as previously described downstream from the reactive particles generator 130. As before, the ozone neutralizing element 160 may be located in any suitable downstream location within the surface treatment device 100. In some embodiments, the downstream location is chosen for ease of access, e.g. to facilitate replacement of the ozone neutralizing element 160 at its end of life as previously explained.

In the above embodiments, the surface treatment device 100 may be a vacuum cleaner, but is not limited thereto. The surface treatment device 100 alternatively may be a device for cleaning soft furnishings, e.g. bedding such as a mattress or any other suitable surface including (human) body surfaces. The air flow generated by the surface treatment device 100 may be too low to effectively collect dirt from a surface contacting the air inlet 105, as previously explained.

The surface treatment device 100 may be manually operated by a user, or alternatively may be a robotic surface treatment device 100 as schematically depicted in FIG. 5. The robotic surface treatment device 100 is typically adapted to autonomously move across a surface to be treated, as is well-known per se. Such a robotic surface treatment device 100 may comprise a controller such as a micro-processor or the like that operates the robotic surface treatment device 100 in accordance with a user-defined or user-selected program for operating the robotic surface treatment device 100.

The robotic surface treatment device 100 may comprise a user interface (not shown) for this purpose, or may comprise a wireless communication unit (not shown) allowing a user of the robotic surface treatment device 100 to wirelessly configure the robotic surface treatment device 100, e.g. using a dedicated remote control or a smart device such as a smart phone, tablet, a laptop computer, a desktop computer and so on having stored thereon an app for generating the appropriate control signals for the robotic surface treatment device 100 and having a controller, e.g. a processor adapted to execute the app to generate the control signals. Such a smart device typically further has wireless communication capability, e.g. a wireless communication module under control of the smart device controller to wirelessly transmit the generated control signals to the robotic surface treatment device 100.

The controller of the robotic surface treatment device 100 may be the controller 155 or a separate controller. The controller of the robotic surface treatment device 100 may be adapted to control a propulsion mechanism of the controller of the robotic surface treatment device 100, e.g. an electromotor driving a set of wheels of the robotic surface treatment device 100 under control of the controller of the robotic surface treatment device 100. The controller of the robotic surface treatment device 100 may be adapted to adjust the propulsion speed and direction of the robotic surface treatment device 100 in accordance with the received user instructions and/or in response to a sensor signal, e.g. indicating air quality as previously explained.

The robotic surface treatment device 100 preferably further comprises a battery or battery pack for providing the necessary electric energy to the various components of the robotic surface treatment device 100 requiring such energy. The battery or battery pack preferably is rechargeable, e.g. through a dedicated charging port of the robotic surface treatment device 100 or through a generic connection such as a universal serial bus connection.

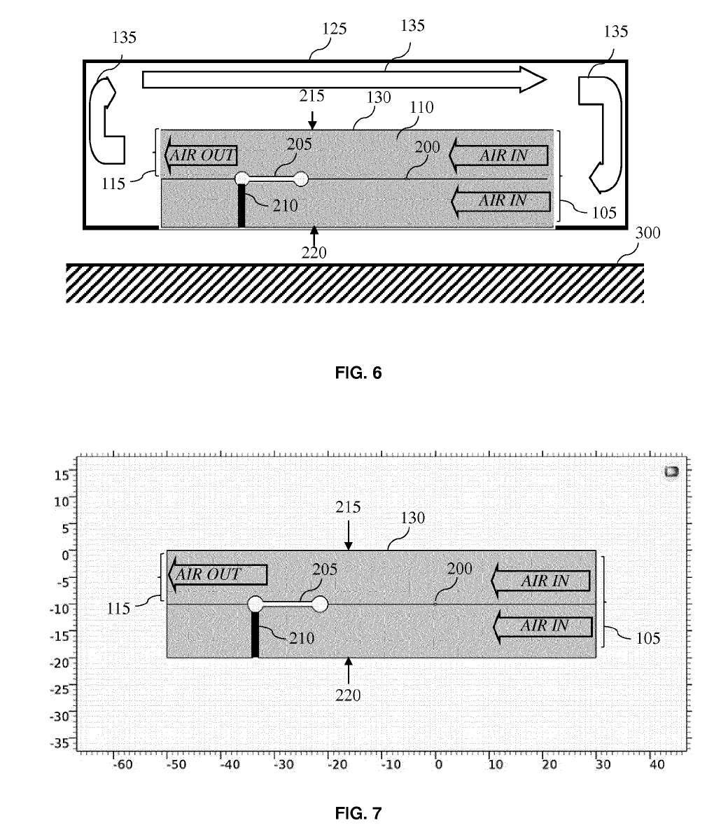

FIG. 6 illustrates an embodiment of a reactive particles generator or ionic wind device (also referred to as ionic wind generator) which generates plasma, and generates an air flow. The ionic wind device comprises an air inlet 105 for supplying air into the ionic wind device. Downstream of the air inlet 105, a corona wire 200 is present for charging incoming air and creating plasma. Downstream of the corona wire 200, a collector electrode 205 is present. Further, an isolating divider 210 is present. The isolating divider 210 is not essential as explained below but advantageous. An air outlet 115 is present downstream of the collector electrode 205 and allows air to exit. The ionic wind device is constructed such that air exiting the air outlet 115 may circulate and re-enter the ionic wind device via the air inlet 105. This circulation is indicated by arrows. The circulating air stream does not contain ions since all ions are captured by the collector electrode 205 but it contains neutral, metastable molecules, like O2(¹Δ) which have a sufficient lifetime in air (˜0.2 s) to survive this “round trip” and will also react with particles on the floor after entering the ionic wind device again. Indicated are also the top 215 and the bottom 220 of the device. When installed in a surface cleaning device, the ionic wind device is positioned such that the side or bottom 220 faces the surface 300 to be cleaned. Thus, the surface cleaning device is constructed such that the bottom 220 of the ionic wind device has direct access to the surface 300. For this purpose, bottom 220 of the ionic wind device contains one or more openings for exposing the surface to the generated plasma, e.g. at the location of the corona wire 200.

### Experimental Data and Results:

FIG. 7 illustrates the dimensions in millimeter of an embodiment of an ionic wind device which may be implemented in a surface cleaning device such as a vacuum cleaner. During simulations a device (=dimension perpendicular to the plotted plane if FIG. 7) having a width of 400 mm was used. The resulting air flow is about 10 m3/hr, the corona (ion) current is about 400 μA and the corona power is about 1.5W. When the device width of 400 mm is scaled and the wire/collector voltages are held constant, then the air flow, the corona current and the corona power will scale proportional to the device width.

The total length of the simulated “ionic wind unit” (=grey shaded area) is 80 mm, the total height is 20 mm. The height of the air inlet 105 (on the rightside in FIG. 7) is 20 mm, and the height of the air outlet 115 (on the leftside in FIG. 7) is 10 mm. For the given situation (=10m3/hr flow for 400 mm device width) these height dimensions should not be reduced by more than 20%, because otherwise the airway resistance would increase too much, leading to a reduced air flow. The width of the “outer” air channels (indicated as 135 in FIG. 6) are preferably at least 1.5 times the height of the air outlet to avoid the build-up of a too large total airway resistance.

The corona wire is designed according the state-of-the-art for ESP devices; e.g. with a diameter as small as possible, e.g. 35 μm, while maintaining mechanical stability and sufficient operational lifetime. The distance between corona wire and the right edge of the collector (here: 20 mm) is determined within a narrow range for efficient operation, this range is 15-25 mm. The distance between corona wire and air inlet 105 (here: 30 mm) is preferably at least 1.5 times the distance between wire and collector, because otherwise a too strong ionic wind opposite to the desired direction of the air flow would develop.

The collector electrode preferably has two rounded edges to avoid too high electric field strengths and risk of breakdown at these edges. For this embodiment the curvature radii of these rounded edges is preferably larger than 1.5 mm. The distance between the edge of the collector and the air outlet, whereby the edge of the collector is the edge of the collector which is the closest to the air outlet, should be at least 15 mm to avoid too high electric field strengths. In this case it is 15 mm. The length of the collector electrode (in this case 15 mm) may be chosen between “4 curvature radii” (in this case 6 mm) and the distance between collector and corona wire (in this case 20 mm).

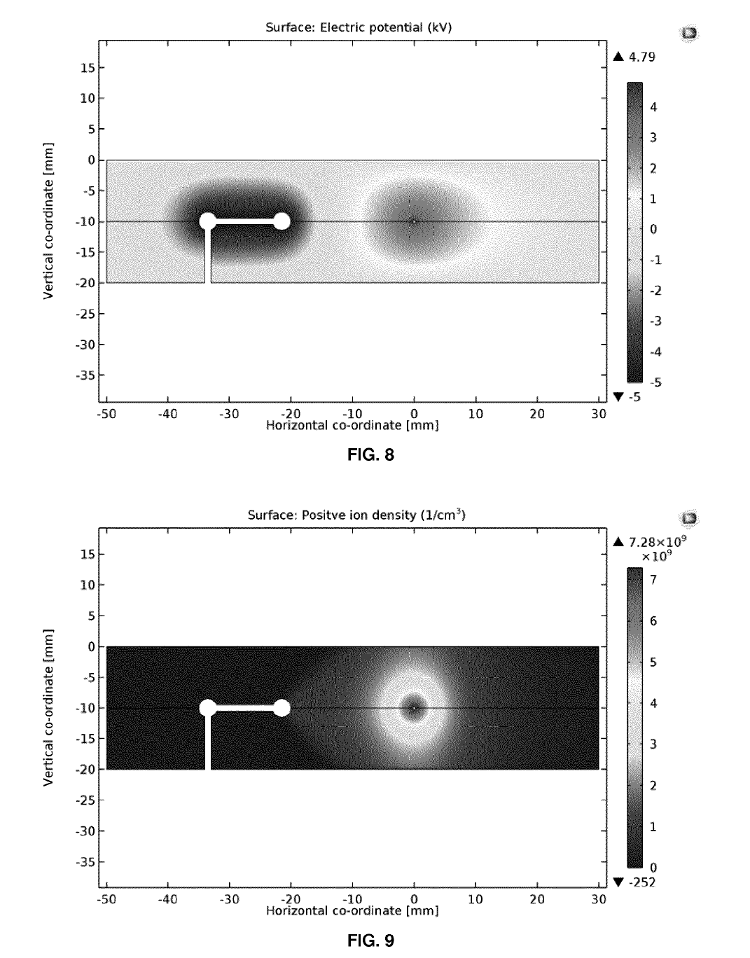

FIG. 8 illustrates the distribution of electric potential inside the ionic wind device illustrated in FIG. 7.

FIG. 9 illustrates the positive ion density u inside the ionic wind device illustrated in FIG. 7.

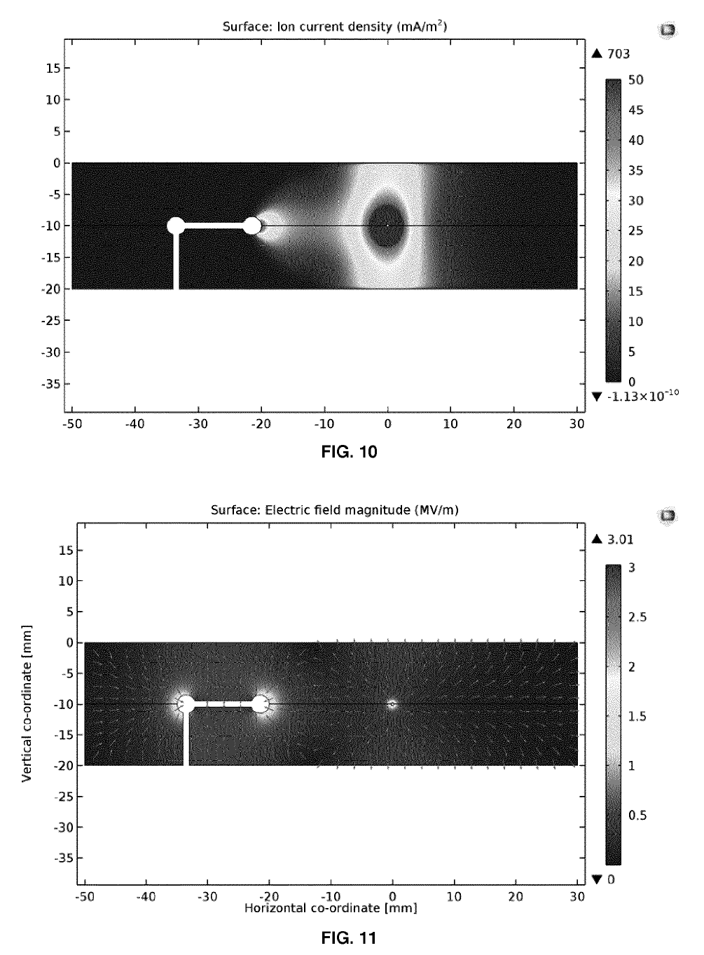

FIG. 10 illustrates the ion current density inside the ionic wind device illustrated in FIG. 7.

FIG. 11 illustrates the electric field strength magnitude at the collector of the ionic wind device illustrated in FIG. 7.

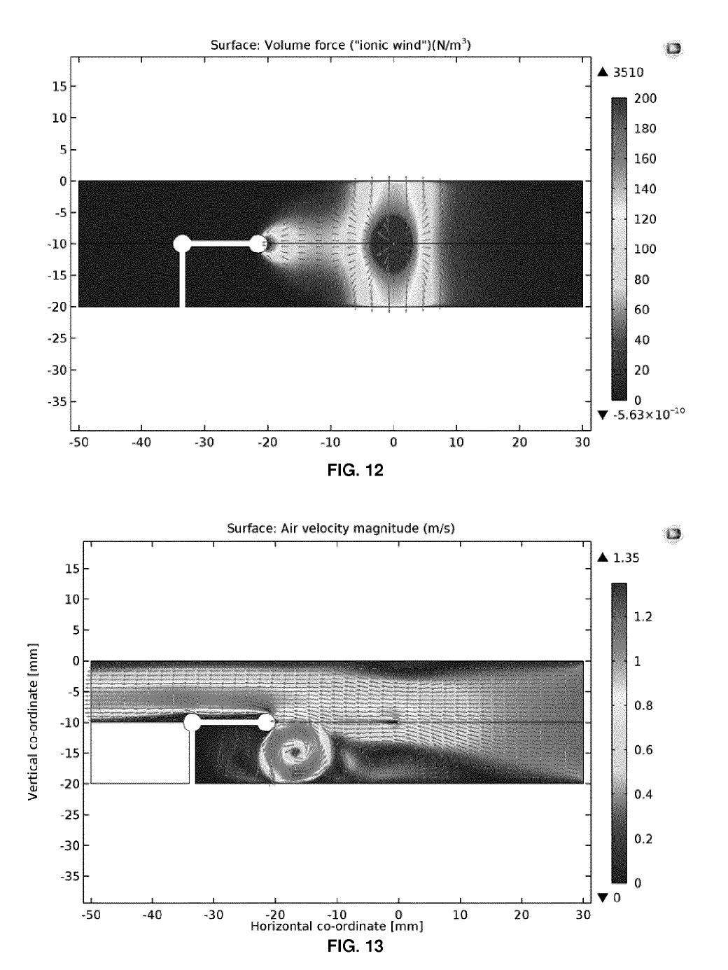

FIG. 12 is a vector plot of the “ionic wind” volume force in the ionic wind device illustrated in FIG. 7. This ionic wind is the driving air flow of the surface cleaning device.

FIG. 13 is a vector plot of the air velocity in the ionic wind device illustrated in FIG. 7.

The following table contains the performance data of the embodiment of the ionic wind device as illustrated in FIG. 7:

| | | |
|---|---|---|
| Corona current density | 700 | mA/m² |
| Corona power | 1.4 | W |
| Corona wire voltage | 5.9 | kV |
| Collector voltage | −5 | kV |
| Air flow | 9.6 | m³/hr |
| Max. E-field at collector | 2.0 | MV/m |
| Max. pos. ion density | 7.3 | E9/cm³ |

As can be noticed in FIG. 10, when the ionic device is activated, a first plasma zone at the corona wire 200 is generated. The first plasma zone stretches down to the bottom 220 and up to the top 215 of the ionic wind device. When the bottom 220 of the ionic wind device is positioned close to or on a surface 300, e.g. a floor, the plasma acts on the particles present on that surface 300. In FIG. 10, this first plasma zone can be noticed between coordinates −8 mm and 8 mm on the horizontal axis and between coordinates −10 mm and −20 mm on the vertical axis. Thus, when a surface treatment device comprising such an ionic device located close the surface is sweeping that surface, the surface is exposed to this plasma zone thereby disinfecting the surface.

Further, in FIG. 10 it can be noticed that at the collector electrode 205 a second plasma zone is present. This zone can be noticed between coordinates −15 mm and −20 mm on the horizontal axis and between coordinates −6 mm and −12 mm on the vertical axis. However, this second plasma zone does not stretch down to the bottom 220 or up to the top 215 of the ionic device.

Surprisingly, it was noticed by the inventors that during air flow generation with the ionic wind device, a vortex is created inside the ionic wind device. The desired vortex is a consequence of the asymmetric design of the “ionic wind unit”. In this embodiment, a part of the device, e.g. the lower half of the device, is closed near the outlet 115 (to the left), by an isolating support of the collector electrode. The creation of this vortex can be noticed in FIG. 13 between coordinates −22 mm and −10 mm on the horizontal axis and between −10 mm and −20 mm on the vertical axis. When the bottom 220 of the ionic wind device is positioned close to a surface, the created vortex stretches down to this surface 300. As a result, particles which are located on the surface 300 are sucked by the vortex into the ionic wind device and are transported to the plasma zone located at the collector electrode 205 where they are exposed to the plasma and thereby neutralized.

It is an important advantage of the invention that the particles on the surface 300 are exposed to a first and a second plasma zone. As an advantage, efficient disinfection of the surface can be obtained.

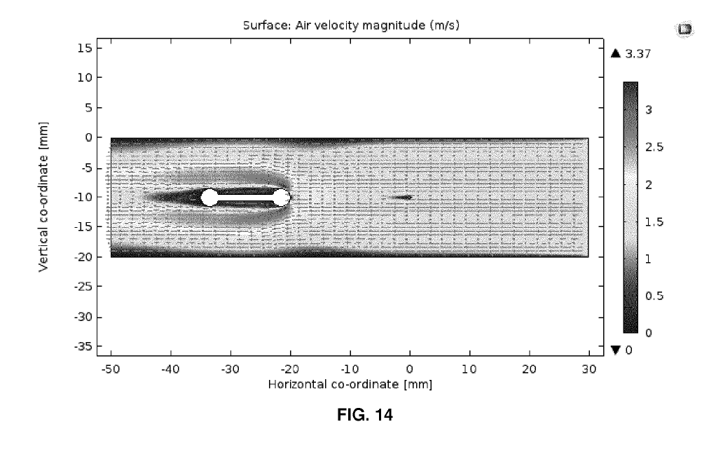

If the isolating support is omitted, a symmetric design is obtained and this vortex is absent. This can be seen in FIG. 14. In summary, this invention presents a surface cleaning device. The surface cleaning device comprises an ionic wind device for exposing a surface to plasma. The ionic wind device is located such that the surface is directly exposed to plasma generated by the ionic wind device. The ionic wind device comprises a conduit 110 with an air inlet 105 and an air outlet 115. These are openings through which air may pass and flow from air inlet 105 to the air outlet 115 via the conduit 110. Inside the conduit 110 a corona wire 200 and a collector electrode 205 are present for creating the ionic wind and generating plasma. When operating the ionic wind device, incoming air is charged and plasma is generated. The ionic wind device may be enclosed in a casing 125 which allows recirculation of air exiting the ionic wind device via the air outlet 115 to the air inlet 105 of the ionic wind device. A side or bottom 220 of the ionic wind device contains one or more openings that allow a surface 300, e.g. a floor, to be directly exposed to the generated plasma when the side 220 is positioned close to the surface 300, e.g. a few centimeters. The casing 125 of the ionic wind device is constructed such that the one or more openings in the side 220 are not blocked and such that the surface 300 can be exposed directly to generated plasma of the ionic device. The ionic wind device may be present in, for example, a cleaning head of the surface cleaning device. For example, the ionic wind device may be present in the cleaning vacuum head of a vacuum cleaner. There it may function to neutralize particles, e.g. allergens, on a surface 300, e.g. a floor, by directly exposing the surface 300 to generated plasma. The ionic wind device may be present in a robot cleaner as illustrated in FIG. 5. The ionic wind device is located in the robot cleaner such that generated plasma of the ionic wind device directly disinfects the floor.

In operation, the ionic wind device features a first zone of plasma generated at the corona wire 200 and a second zone of plasma at the collector electrode 205, see FIG. 10. The dimensions of the ionic wind device and the powering of the corona wire 200 are selected such that generated plasma at the corona wire 205 stretches down to the side 220 of the ionic device that is positioned close to the surface 300 that is to be cleaned. When placing the ionic wind device close to the surface 300, for example by placing the vacuum cleaning head close to the surface (e.g. a few centimeters, e.g. less than 5 centimeters, e.g. less than 2 centimeters, e.g. less than 1 centimeter), the surface is directly exposed to the generated plasma from the first plasma zone. This means that the generated plasma must not be transported first to expose the surface 300 to the generated plasma. This eliminates the number of parts required and thereby reduces cost.

The ionic wind device may feature an internal asymmetric design. For example, inside the ionic wind device an isolating divider 210 may be present. The isolating divider 210 may be a part located and formed in the ionic wind device (thus, in conduit 110) to partly block the air flow inside the ionic wind device thereby creating a vortex. The isolating divider 210 may be present in one half of the ionic wind device. For example, a lower half of the device wherein the lower half is defined as the half of the device which is closest to a surface 300 when the ionic wind device is positioned on that surface 300. The internal asymmetric design is constructed such that inside the ionic wind device a vortex is created when air flows from the air inlet 105 to the air outlet 115. Further, the internal asymmetric design is constructed such that, when in operation, the vortex stretches down to the surface 300 which is to be cleaned and picks up particles, e.g. allergens, from that surface 300 and transports the particles to the second plasma zone at the collector electrode 205. See FIG. 13. The asymmetric design proves to be a very effective manner of disinfecting a surface. The ionic device and the casing are constructed, e.g. featuring appropriate openings, to allow particles on the surface 300 to be directly disinfected by 1) generated plasma at the corona wire 200 and by 2) generated plasma at the counter electrode 205 through transportation of particles from the surface 300 to that generated plasma at the counter electrode 205 via a created vortex inside the ionic wind device. Both disinfection manners are possible when the ionic device is positioned close to the surface 300, e.g. a centimeter or less.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. For example, as mentioned above, claim 1 covers an embodiment in which the reactive particles generator 130 and the air flow generator 150 are formed by a single unit (e.g. a corona discharge generator) for generating an ionic wind from a surface, the ionic wind having a net air flow velocity of 1 m/s or less, to subject the surface to the ionic wind. The mere fact that certain measures are recited in mutually different dependent claims does not indicate that a combination of these measures cannot be used to advantage.

1. A surface treatment device for neutralizing allergens on a surface, comprising:
an air inlet;
an air outlet;
a conduit located in between the air inlet and the air outlet;
an air flow generator for generating an air flow from the air inlet to the air outlet through the conduit;
wherein the conduit comprises a reactive particles generator for generating reactive particles from air;
wherein:
the reactive particles generator is arranged to subject the surface to the generated reactive particles; wherein the reactive particles generator is a corona discharge device; and wherein the corona discharge device is configured to act as the air flow generator by generating ionic wind; and wherein the reactive particles generator comprises a corona wire, and wherein the conduit and the corona wire are adapted such that the particles on the surface are directly exposed to the generated reactive particle at the corona wire when the surface is subjected to the surface treatment device.

2. (canceled)

3. The surface treatment device according to claim 1, wherein the reactive particles generator further comprises a collector electrode, and wherein the conduit and the collector electrode are adapted to generate a vortex inside the conduit when the air flow is generated such that particles on the surface are transported towards generated plasma at the counter electrode via the vortex when the surface is subjected to the surface treatment device.

4. The surface treatment device according to claim 3, wherein the conduit features an isolating divider supporting the collector electrode, the isolating divider being positioned inside the conduit and adapted for generating the vortex.

5. The surface treatment device according to claim 1, wherein the surface treatment device is fan-less.

6. The surface treatment device according to claim 1,
wherein the air flow generator is controlled by a controller to control the air flow rate produced by the air flow generator, and
wherein the air flow generator is adapted such that the air flow is in the range of 0-10 m³/hour.

7. The surface treatment device according to claim 1,
wherein the air flow generator is controlled by a controller to control the air flow rate produced by the air flow genetor; and
wherein the air flow generator is adapted for generating an air flow having an air flow velocity of 1 m/s or less.

8. The surface treatment device according to claim 1, further comprising an ozone neutralizing element located downstream from the reactive particle generator.

9. The surface treatment device according to claim 8, wherein the ozone neutralizing element comprises an active carbon containing element or a catalyst for neutralizing ozone.

10. The surface treatment device according to claim 1, comprising a cleaning head including the reactive particles generator.

11. The surface treatment device according to claim 1, wherein the surface treatment device is a vacuum cleaner.

12. The surface treatment device according to claim 1, wherein the reactive particles generator located in a treatment chamber which is in fluid connection with or as part of the conduit.

13. The surface treatment device according to claim 12, wherein the air inlet and the air outlet are in fluid connection with the treatment chamber thereby creating recirculation of air around the reactive particle generator in the treatment chamber.

14. A method for neutralizing allergens on a surface, comprising:
generating an air flow from a surface;
generating reactive particles from air;
subjecting the surface to the reactive particles thereby neutralizing allergens present on the surface;
wherein:
generating the air flow is performed by generating an ionic wind and subjecting the surface to the ionic wind.

15. A method as claimed in claim 14, wherein the generated ionic wind has an air flow velocity of 1 m/s or less.
